(19) United States
(12) Patent Application Publication
(10) Pub. No.: US 2023/0218152 A1
(43) Pub. Date: Jul. 13, 2023

(54) ENDOSCOPE AND ENDOSCOPE PRODUCING METHOD

(71) Applicant: OLYMPUS MEDICAL SYSTEMS CORP., Tokyo (JP)

(72) Inventor: Takahiro SUWA, Tokyo (JP)

(73) Assignee: OLYMPUS MEDICAL SYSTEMS CORP., Tokyo (JP)

(21) Appl. No.: 18/093,896

(22) Filed: Jan. 6, 2023

Related U.S. Application Data
(60) Provisional application No. 63/298,296, filed on Jan. 11, 2022.

(51) Int. Cl. A61B 1/06; A61B 1/00

(52) U.S. Cl. CPC A61B 1/0676; A61B 1/0011

(57) ABSTRACT

An insertion portion of an endoscope including a distal end portion including: an exterior surface, a first channel including a first opening on the exterior surface, and a second channel including a second opening on the exterior surface and a third opening on the first channel. An elongated object is inserted into the first channel. A rigid portion is provided at a distal end of the first elongated object. The rigid portion including: an outer peripheral surface, and a first surface recessed from the outer peripheral surface of the rigid portion. Wherein both of the outer peripheral surface and the first surface are exposed within the third opening.

### CROSS-REFERENCE TO RELATED APPLICATION

This application is based on and claims priority under 35 U.S.C. § 119 to U.S. Provisional Application No. 63/298,296, filed Jan. 11, 2022, the entire contents of which is incorporated herein by reference.

### BACKGROUND

### 1. Technical Field

The present disclosure relates to an endoscope and an endoscope producing method.

### 2. Related Art

As an endoscope, there is known a side-viewing endoscope that observes a subject from a direction intersecting an insertion axis direction of an insertion portion to be inserted into the subject. In this side-viewing endoscope, built-in objects such as a side-viewing optical system having an illumination unit for illuminating a subject and an imaging unit for capturing an image of the subject are provided at the distal end portion of the insertion portion.

The built-in object includes a built-in object having a bent portion bent in the middle along the insertion axis direction, and having, with the bent portion as a boundary, a first portion located on the distal end side and formed along a direction intersecting the insertion axis, and a second portion located on the proximal end side and extending in the insertion axis direction. In the first portion, a rigid portion for fixing to the distal end portion is formed on a peripheral surface. In the following description, for convenience of description, a built-in object having a bent portion will be referred to as a bent built-in object.

In general, a bent built-in object is attached to the distal end portion of the insertion portion as illustrated below.

First, a first portion of the bent built-in object is inserted into a first opening formed in the distal end portion. Positioning is then performed by gripping a second portion of the bent built-in object with tweezers or others and rotating or pushing the second portion. A screw is inserted into a second opening formed in the distal end portion and communicating with the first opening described above, and the rigid portion is pressed and fixed by the screw.

However, since the first portion is inserted into the first opening of the distal end portion, when the second portion is gripped and rotated or pushed, a force is concentrated on the second portion or the bent portion, and the second portion or the bent portion may be damaged.

JP 6704872 B2 discloses that a distal end portion is formed into two bodies of a main body block and a moving block, a bent built-in object is positioned in advance with respect to the moving block having a first opening for holding a first portion, and then the main body block and the moving block are fixed to each other.

### SUMMARY

Accordingly, an insertion portion of an endoscope is provided. The insertion portion comprising: a distal end portion comprising: an exterior surface, a first channel including a first opening on the exterior surface, and a second channel including a second opening on the exterior surface and a third opening on the first channel, an elongated object inserted into the first channel; a rigid portion provided at a distal end of the first elongated object, the rigid portion including: an outer peripheral surface, and a first surface recessed from the outer peripheral surface of the rigid portion, wherein both of the outer peripheral surface and the first surface are exposed within the third opening.

The rigid portion can comprise a second surface recessed from the outer peripheral surface. The first surface and the second surface can be separated from each other in a circumferential direction of the rigid portion.

The first surface can include a side surface and a bottom surface, the side surface and the bottom surface can form an angle of 90° to 135° relative to each other.

The insertion portion can further comprise a fixing portion inserted into the second channel, the fixing portion can contact the outer peripheral surface so as to hold the rigid portion. The fixing portion can be configured to press the outer peripheral surface of the rigid portion. The fixing portion can include a distal end having a size greater than a size of an opening of the recessed surface on the outer peripheral surface.

The first elongated object can include a distal portion at a distal-end side that is bent relative to a proximal portion of the first elongated object, and the rigid portion can be provided on the distal portion. The first elongated object can be an illumination unit configured to emit light for illuminating a subject. The illumination unit can be a light guide.

The exterior surface can include an outer surface, a distal end surface and an inclined surface between the outer surface and the distal end surface, wherein the first opening can be provided on the inclined surface, and the second opening can be provided on the outer surface.

A first longitudinal axis of the first channel can intersect with a second longitudinal axis of the second channel.

The second channel can extend linearly.

The rigid portion can be configured to satisfy a relationship of W>d+r, where W is a width dimension of the second opening, r is a width dimension of each of the first surface and the second surface, and d is a distance between the first surface and the second surface.

The rigid portion can be formed of a first material having a rigidity greater than a rigidity of a second material forming the elongated object.

The rigid portion can include: a raised surface adjacent to one or more of the first surface and the second surface, and a transition surface separate from the first surface and the raised surface, the transition surface forming a transition between the raised surface and one or more of the first surface and the second surface, wherein at least a portion of each of the recessed surface, the raised surface and the transition surface are exposed within the third opening.

Also provided is an endoscope comprising: the insertion portion as described above.

Still yet provided is a method of assembling an insertion portion of an endoscope. The method comprising: inserting a rigid portion onto a distal end of an elongated object; inserting the elongated object into a first channel of a distal end portion; positioning the rigid portion to a first opening of the first channel, the first opening communicating the first channel with a second channel of the distal end portion; positioning one of a first surface recessed from an outer peripheral surface of the rigid portion at the first opening; inserting an adjustment tool into the second channel; and positioning of the elongated object with respect to the distal end portion by applying a load to the first surface through the second channel with the adjustment tool.

The method can further comprise positioning an outer peripheral surface of the rigid portion at the first opening.

Still further yet provided is an insertion portion of an endoscope. The insertion portion comprising: a distal end portion comprising: an exterior surface, a first channel including a first opening on the exterior surface, and a second channel including a second opening on the exterior surface and a third opening on the first channel, an elongated object inserted into the first channel; a rigid portion provided at a distal end of the first elongated object, the rigid portion including: a recessed surface, a raised surface adjacent to the recessed surface; and a transition surface separate from the recessed surface and the raised surface, the transition surface forming a transition between the recessed surface and the raised surface; wherein at least a portion of each of the recessed surface, the raised surface and the transition surface are exposed within the third opening.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram for explaining a configuration of an endoscope according to an embodiment;

FIG. 2 is a diagram for explaining a configuration of a distal end portion;

FIG. 3 is a cross-sectional view of a second distal end component portion taken along a plane passing through an illumination window and a machined hole and including an insertion axis of an insertion portion;

FIG. 4 is a diagram for explaining a configuration of a rigid portion;

FIG. 5 is a diagram of the inside of a machined hole viewed along an arrow A in FIG. 3;

FIG. 6 is a diagram for explaining a method for producing an endoscope;

FIG. 7 is a diagram for explaining the method for producing the endoscope;

FIG. 8 is a diagram for explaining the method for producing the endoscope;

FIG. 9 is a diagram for explaining a first modification of the embodiment;

FIG. 10 is a diagram for explaining the first modification of the embodiment;

FIG. 11 is a diagram for explaining a second modification of the embodiment;

FIG. 12 is a diagram for explaining the second modification of the embodiment;

FIG. 13 is a diagram for explaining a third modification of the embodiment;

FIG. 14 is a diagram for explaining the third modification of the embodiment; and

FIG. 15 is a diagram for explaining the third modification of the embodiment.

### DETAILED DESCRIPTION

Modes for carrying out the disclosure (hereinafter, referred to as “embodiments”) will be described below with reference to the drawings. The disclosure is not limited by this embodiment. Further, in the drawings, the same portions are denoted by the same reference numerals. Further, it is necessary to note that the drawings are schematic illustration in which the relationships between the thickness and the width of each member, and the proportions of each member, for example, may differ from the actual relationships and proportions. In addition, there may be differences in dimensions and proportions between the drawings.

### Embodiments

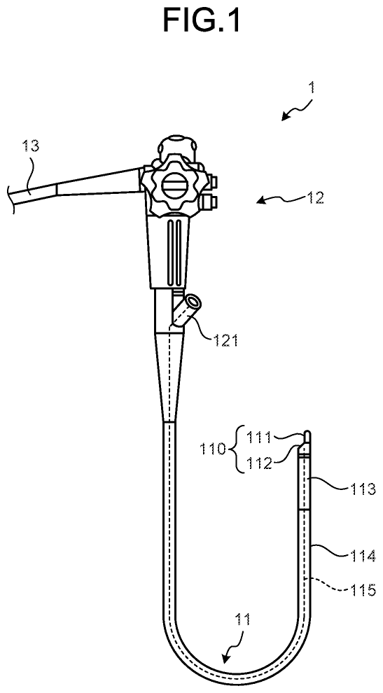

An endoscope and a method for producing an endoscope according to an embodiment will be described with reference to FIGS. 1 to 8.

### Configuration of Endoscope

FIG. 1 is a diagram for explaining a configuration of an endoscope 1 according to the embodiment.

The endoscope 1 illustrated in FIG. 1 is an ultrasound endoscope that transmits ultrasound waves to a subject that is an observation target and receives ultrasound waves reflected by the subject. Note that, although the ultrasound endoscope has been described as an example of an endoscope, the endoscope may be an endoscope having no ultrasound probe, such as a duodenoscope.

Note that a “distal end” described below means an end portion on an insertion direction side into a subject. A “rear end” (or “proximal end”) means an end portion on the opposite direction side to the insertion direction into the subject.

As illustrated in FIG. 1, the endoscope 1 includes an insertion portion 11, an operating unit 12, and a universal cord 13.

The insertion portion 11 has a long shape to be inserted into a subject. The insertion portion 11 includes a distal end portion 110 made of resin or others, a bending portion 113 configured to be bendable in accordance with an operation input received by the operating unit 12, and a flexible tube portion 114 having flexibility. A treatment tool channel 115, which is a conduit through which a treatment tool such as forceps or a puncture needle is inserted, is formed inside the insertion portion 11. The distal end portion 110 has an exterior surface.

The operating unit 12 is provided at the rear end of the insertion portion 11. The operating unit 12 is held by a user and also receives an operation input by the user. The operating unit 12 includes a treatment tool insertion port 121 for inserting a treatment tool into the treatment tool channel 115. A treatment tool inserted from the treatment tool insertion port 121 can pass through the treatment tool channel 115, protrude from the distal end portion 110 to the outside, and access the inside of the body via the endoscope 1.

The universal cord 13 extends from the operating unit 12. The universal cord 13 is provided with a connector (not illustrated) at an end portion opposite to the operating unit 12, and is connected to peripheral devices (not illustrated) such as an ultrasound observation device, a camera control unit, a display device, an air/water supply device, and a light source device via the connector.

### Configuration of Distal End Portion

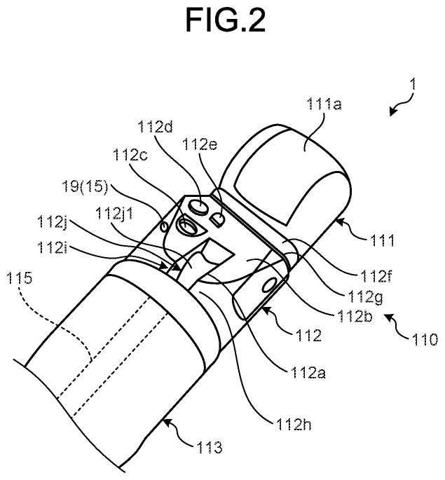

FIG. 2 is a diagram for explaining a configuration of the distal end portion 110.

As illustrated in FIG. 2, the distal end portion 110 includes a first distal end component portion 111 on the distal-end side and a second distal end component portion 112 (distal end main body) on the rear (or proximal) end side. The first distal end component portion 111 includes a convex-type ultrasound probe 111a that transmits and receives ultrasound waves to and from a subject. Although the ultrasound probe 111a is a convex type, the ultrasound probe is not limited to the convex type and may be a linear type or a radial type.

The second distal end component portion 112 includes a treatment tool lead-out port 112a, an inclined portion 112b, an observation window 112c, an illumination window 112d, an air/water supply nozzle 112e, a first coupling member 112f, a balloon groove 112g, a second coupling member 112h, a raising base housing portion 112i, a raising base 112j, and a machined hole 15 (second hole).

The treatment tool lead-out port 112a is an opening through which a treatment tool inserted through the treatment tool channel 115 protrudes. The treatment tool lead-out port 112a includes the raising base housing portion 112i formed to have a concave shape when the second distal end component portion 112 is viewed from the distal-end side. The raising base housing portion 112i is a space for which the raising base 112j is used. The raising base housing portion 112i is formed so as to communicate with the treatment tool channel 115. Therefore, when the treatment tool inserted through the treatment tool channel 115 reaches the treatment tool lead-out port 112a, the treatment tool protrudes from the treatment tool lead-out port 112a to the outside. The raising base 112j is held rotatably with respect to the second distal end component portion 112. When the operating unit 12 receives an operation input by the user, the raising base 112j is rotated and the direction of the distal end is changed. In other words, in a state where the treatment tool is inserted through, the protruding direction of the treatment tool can be changed by a guide surface 112j1 of the raising base 112j.

The inclined portion 112b is formed so as to be inclined with respect to the insertion axis of the insertion portion 11 (the longitudinal axis of the insertion portion 11).

External light is incident on the observation window 112c. The observation window 112c is formed of a light-transmitting member such as a transparent resin. The light emitted from the illumination window 112d impinges on a subject, and light such as scattered light is incident on the observation window 112c. An imaging unit including a lens and an image sensor (not illustrated) is disposed inside the second distal end component portion 112. The light incident from the observation window 112c forms an image on the image sensor and is output to a peripheral device. The image sensor is an optical sensor such as a charge coupled device (CCD) or a complementary metal oxide semiconductor (CMOS), but may be a sensor other than the optical sensor such as a temperature sensor or a speed sensor.

The illumination window 112d emits light to the outside. The illumination window 112d is formed of a light-transmitting member such as a transparent resin. The illumination window 112d faces the distal end of a light guide 14 disposed inside the universal cord 13, the operating unit 12, and the insertion portion 11. The light guided by the light guide 14 is emitted from the illumination window 112d toward the subject to illuminate the subject. Although it has been described that the light emitted from the illumination window 112d is the light guided by the light guide 14, a light emission diode (LED) may be disposed inside the second distal end component portion 112 so that the LED emits light.

The air/water supply nozzle 112e is a nozzle through which a cleaning liquid or a gas for cleaning the observation window 112c flows. The air/water supply nozzle 112e is disposed such that an opening on the distal-end side faces the observation window 112c. The air/water supply nozzle 112e has an opening on the rear end side connected to the air/water supply device via a conduit (not illustrated) disposed inside the universal cord 13, the operating unit 12, and the insertion portion 11.

The first coupling member 112f is a tubular member that couples the first distal end component portion 111 and the second distal end component portion. Inside the first coupling member 112f, a signal cable (not illustrated) connected to the ultrasound probe 111a, an insulating pipe covering the signal cable, and others are housed.

The balloon groove 112g is an annular groove provided on the outer periphery of the first coupling member 112f. The balloon groove 112g fixes a balloon (not illustrated). The balloon (not illustrated) covers the ultrasound probe 111a.

The second coupling member 112h couples the bending portion 113 and the second distal end component portion 112. The second coupling member 112h is a non-inclined portion provided in the second distal end component portion 112 and has a tubular shape extending from the rear end side toward the distal-end side. The second coupling member 112h is disposed on the rear end side of the inclined portion 112b, and the treatment tool lead-out port 112a is located in a part of the second coupling member 112h.

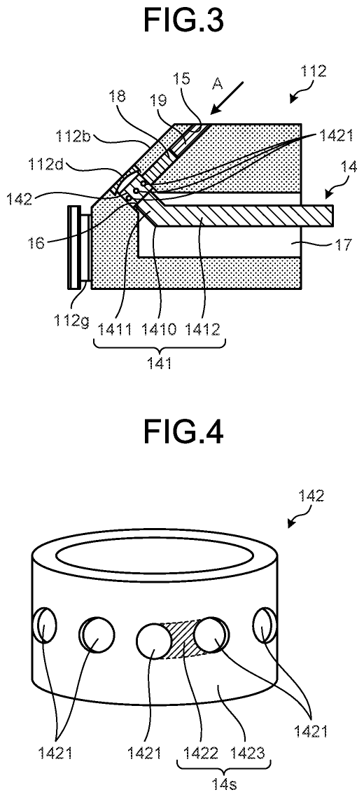

FIG. 3 is a cross-sectional view of the second distal end component portion 112 taken along a plane passing through the illumination window 112d and the machined hole 15 and including the insertion axis of the insertion portion 11.

As illustrated in FIG. 3, the second distal end component portion 112 has, inside of the portion, a holding hole 16 (first hole) and a housing hole 17 that house the light guide 14. The housing hole 17 is a hole extending from the rear end of the second distal end component portion 112 toward the distal end along the insertion axis of the insertion portion 11. The holding hole 16 is a hole communicating with the distal end of the housing hole 17 and extending from the distal end of the housing hole 17 to the inclined portion 112b. The illumination window 112d is provided at an opening on the distal-end side of the holding hole 16.

The housing hole 17 (first channel) includes a first opening on the exterior surface, and the machined hole 15 (second channel) includes a second opening on the exterior surface and a third opening on the first channel 17. An elongated object 14 inserted into the first channel. The elongated object 14 is at least one of the light guide, an imaging unit, an air supply or suction channel and a water supply or suction channel. The exterior surface includes an outer surface, which can be an outer peripheral surface including an outer circumferential surface, a distal end surface and an inclined surface between the outer surface and the distal end surface, the first opening is provided on the inclined surface, and the second opening is provided on the outer surface. A first longitudinal axis of the first channel 17 intersects with a second longitudinal axis of the second channel 15. The second channel 15 extends linearly.

The machined hole 15 is a hole for positioning a first portion 1411 of the light guide 14 (built-in object), which will be described below, with respect to the second distal end component portion 112. The machined hole 15 extends from the side surface of the second distal end component portion 112 toward the inside of the second distal end component portion 112 and communicates with the holding hole 16. More specifically, the machined hole 15 has a circular cross-sectional shape extending in a direction intersecting the direction in which the holding hole 16 extends. Note that the machined hole 15 can be provided at a position where the machined hole does not interfere with a built-in object held by the second distal end component portion 112 other than the light guide 14.

A screw 18 (fixing portion) and a lid member 19 are inserted into the machined hole 15. The screw 18 fixes the light guide 14 in a state of being housed in the holding hole 16 and the housing hole 17. The lid member 19 is configured to close the machined hole 15.

The light guide 14 is constituted of a light guide main body 141 and a rigid portion 142. The light guide main body 141 includes a bent portion 1410 that is bent in the middle along the longitudinal axis, a first portion 1411 that is on the distal-end side of the bent portion 1410, and a second portion 1412 that is on the rear end side of the bent portion 1410. The bent portion 1410 is formed in a state of being bent in advance so that the light guide 14 maintains a predetermined shape.

FIG. 4 is a diagram for explaining a configuration of the rigid portion 142.

The rigid portion 142 is formed of a member that is more rigid than the light guide main body 141, such as metal. That is, the rigid portion 142 is formed of a first material having a rigidity greater than a rigidity of a second material forming the elongated object 14. As illustrated in FIG. 4, the rigid portion 142 is an annular member that covers the periphery of the first portion 1411. A plurality of adjustment holes 1421 (hole portions (alternatively referred to first surface, second surface and/or recessed surface) having circular openings is formed on an outer surface 14s of the rigid portion 142, which can be an outer peripheral surface including an outer circumferential surface. In the present embodiment, the plurality of adjustment holes 1421 are arranged in a row along the circumferential direction of the rigid portion 142 (the circumferential direction about the longitudinal axis of the light guide main body 141) while being spaced apart from each other by a separation distance d, which will be described below. In other words, the outer peripheral surface 14s is distinguished into a boundary portion 1422 between the adjustment holes 1421 adjacent to each other, and a peripheral wall 1423 other than the boundary portion. Note that, in FIG. 4, for convenience of description, only one boundary portion 1422 among the plurality of boundary portions 1422 is represented by oblique lines.

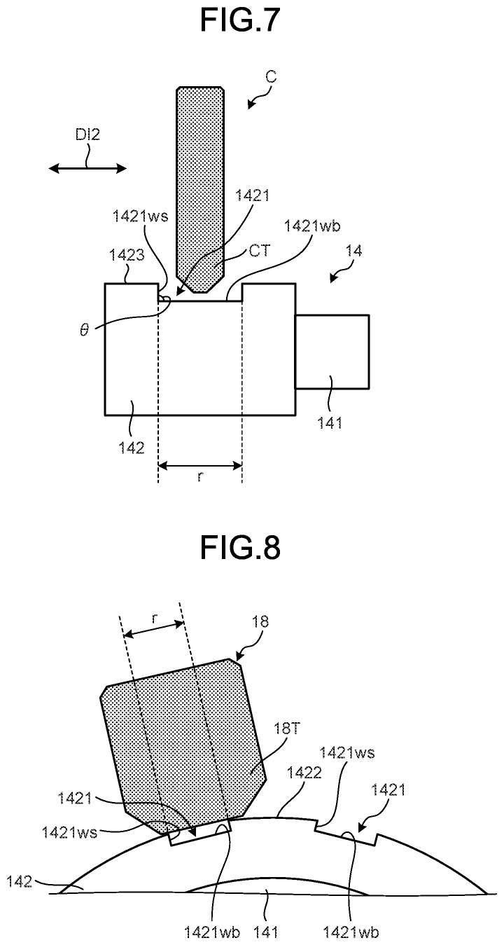

The rigid portion 142 is provided at a distal end of the first elongated object 14. The rigid portion includes an outer peripheral surface 1422 or 1423, and a first surface (adjustment hole) 1421 recessed from the outer peripheral surface 1422 or 1423 of the rigid portion 142. Although such first surface can be any surface of the adjustment hole 1421, more specifically, as described below, the first surface can be side surface 1421ws and/or bottom 1421wb (see e.g., FIG. 7). Both of the outer peripheral surface 1422 or 1423 and the first surface 1421 are exposed within the third opening. The rigid portion 142 comprises a second surface 1421 recessed from the outer peripheral surface 1422 or 1423. The first surface 1421 and the second surface 1421 are separated from each other in a circumferential direction of the rigid portion 142.

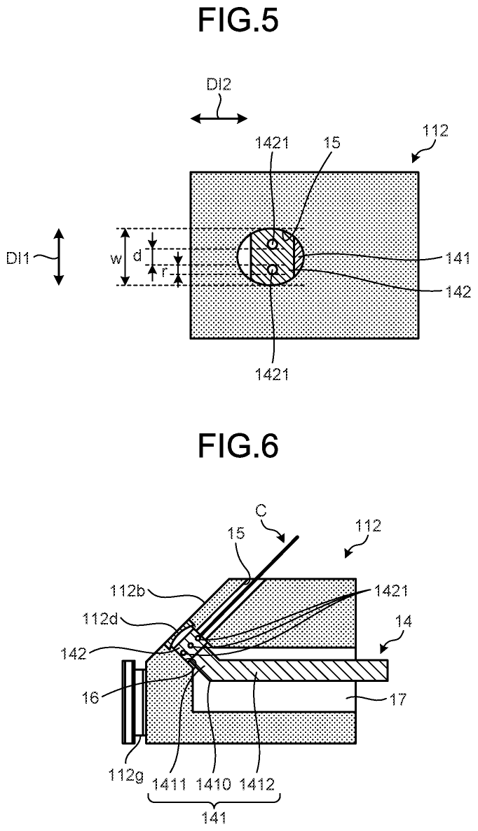

FIG. 5 is a diagram of the inside of the machined hole 15 viewed along an arrow A in FIG. 3 with respect to the second distal end component portion 112. Note that, for convenience of explanation, FIG. 5 illustrates a state in which the screw 18 and the lid member 19 are not provided. FIG. 5 illustrates a state after the light guide 14 is positioned with respect to the second distal end component portion 112 by a method for producing the endoscope 1, which will be described below. In FIG. 5, an arrow DI1 (an arrow indicating the up/down direction in FIG. 5) is a first direction corresponding to the circumferential direction of the rigid portion 142 (the circumferential direction about the longitudinal axis of the light guide main body 141). An arrow DI2 (an arrow indicating the left/right direction in FIG. 5) is a second direction corresponding to the longitudinal axis direction of the light guide main body 141.

As illustrated in FIG. 5, the two adjustment holes 1421 and the boundary portion 1422 face the machined hole 15 in a state after the light guide 14 is positioned with respect to the second distal end component portion 112 by the method for producing the endoscope 1, which will be described below. In other words, it is configured to have a relationship of W>d+r, where W is the width dimension of the machined hole 15 in the first direction DI1 (the diameter dimension of the machined hole 15), r is the width dimension of the adjustment hole 1421 in the first direction DI1 (the diameter dimension of the adjustment hole 1421), and d is the separation distance between the adjustment holes 1421 adjacent to each other in the first direction DI1.

### Method for Producing Endoscope

The method for producing the endoscope 1, such as, a method for positioning the light guide 14 with respect to the second distal end component portion 112 will now be described with reference to FIGS. 6 to 8.

FIGS. 6 to 8 are diagrams for explaining the method for producing the endoscope 1. Specifically, FIG. 6 is a cross-sectional view corresponding to FIG. 3, and is a diagram for explaining a method for positioning using an adjustment tool C. FIG. 7 is a diagram of the adjustment tool C and the rigid portion 142 viewed from a direction orthogonal to the longitudinal axis of the light guide main body 141. FIG. 8 is a diagram of the screw 18 and the rigid portion 142 viewed along the first direction DI1.

The operator first inserts the first portion 1411 of the light guide 14 into the holding hole 16 of the second distal end component portion 112 (step S1). The method comprises inserting the rigid portion 142 onto the distal end of the elongated object 14, inserting the elongated object 14 into the first channel 14 of a distal end portion, positioning the rigid portion 142 to the first opening of the first channel 14, positioning one of the first surface 1421 recessed from the outer peripheral surface 1422 or 1423 of the rigid portion 142 at the first opening, positioning an outer peripheral surface 1422 or 1423 of the rigid portion 142 at the first opening, inserting the adjustment tool C into the second channel 15, and positioning of the elongated object 14 with respect to the distal end portion by applying a load to the first surface 1421 through the second channel 15 with the adjustment tool C.

In this state, the operator inserts the adjustment tool C, which is a long member such as tweezers, from the outside of the machined hole 15 (FIG. 6). The adjustment hole 1421 has a size into which the distal end CT of the adjustment tool C can be inserted (FIG. 7). The size of the adjustment hole 1421 can be set such that the distal end CT of the adjustment tool C can contact a bottom 1421wb of the adjustment hole 1421. Further, a side surface 1421ws constituting the adjustment hole 1421 can be a surface formed so as to form an angle θ of 90° or more and 135° or less with respect to the bottom 1421wb. In the present embodiment, the adjustment hole 1421 is a hole linearly extending toward the central axis of the rigid portion 142. In other words, the angle θ is substantially 90°.

The first surface 1421 includes one or more of the side surface 1421ws and the bottom surface 1421wb, the side surface and the bottom surface form the angle θ of 90° to 135° relative to each other. The first elongated object 14 includes a distal portion at a distal-end side that is bent relative to a proximal portion of the first elongated object 14, and the rigid portion is provided on the distal portion.

The operator inserts the distal end CT of the adjustment tool C into the adjustment hole 1421, and performs positioning by moving the light guide 14 with respect to the second distal end component portion 112 using the adjustment tool C (step S2). Specifically, the operator rotates the light guide 14 about the optical axis with respect to the second distal end component portion 112 to perform positioning of the light guide by pushing the side surface 1421ws constituting the adjustment hole 1421 in the first direction DI1 with the distal end CT. The operator moves the light guide 14 in the optical axis direction with respect to the second distal end component portion 112 to perform positioning of the light guide by pushing the side surface 1421ws in the second direction DI2 with the distal end CT.

After positioning the light guide 14 at a predetermined position, the operator removes the adjustment tool C from the machined hole 15 and inserts the screw 18 instead. The operator presses and fixes the rigid portion 142 with the screw 18 (step S3). A distal end 18T of the screw 18 has a size incapable of being inserted into the adjustment hole 1421 (FIG. 8). In other words, in step S3, the distal end 18T is not inserted into the adjustment hole 1421, but presses the outer peripheral surface 14s to fix the rigid portion 142. Further, the operator inserts the lid member 19 into the machined hole 15 such that the screw 18 is hidden, and fixes the lid member with an adhesive or others (step S4). The fixing portion 18 includes a distal end having a size greater than a size of an opening of the recessed surface 1421 on the outer peripheral surface 1422 or 1423.

According to the present embodiment described above, at least the following effects are obtained.

In the method for producing the endoscope 1 according to the present embodiment, the rigid portion 142 (the first portion 1411) can be directly moved by the adjustment tool C. Therefore, no load is applied to the bent portion 1410 and the second portion 1412, and it is possible to prevent the light guide 14 from being damaged in the producing process.

The rigid portion 142 is also configured to have a relationship of W>d+r, where W is the width dimension of the machined hole 15 in the first direction DI1 (the diameter dimension of the machined hole 15), r is the width dimension of the adjustment hole 1421 in the first direction DI1 (the diameter dimension of the adjustment hole 1421), and d is the separation distance between the adjustment holes 1421 adjacent to each other in the first direction DI1. Therefore, even when the side surface 1421ws constituting one adjustment hole 1421 is pushed in the first direction DI1 by the adjustment tool C to rotate the light guide 14 about the optical axis, the other adjustment hole 1421 adjacent to the one adjustment hole 1421 is always exposed from the machined hole 15. In other words, the light guide 14 can be further rotated about the optical axis by using the other adjustment hole 1421. Therefore, the amount of positioning about the optical axis in the light guide 14 can be increased, which allows good positioning.

Further, the side surface 1421ws constituting the adjustment hole 1421 is a surface formed so as to form an angle θ of 90° or more and 135° or less with respect to the bottom 1421wb. Therefore, the distal end CT of the adjustment tool C is easily caught by the side surface 1421ws. Therefore, good positioning of the light guide 14 with respect to the second distal end component portion 112 using the adjustment tool C can be performed.

### First Modification

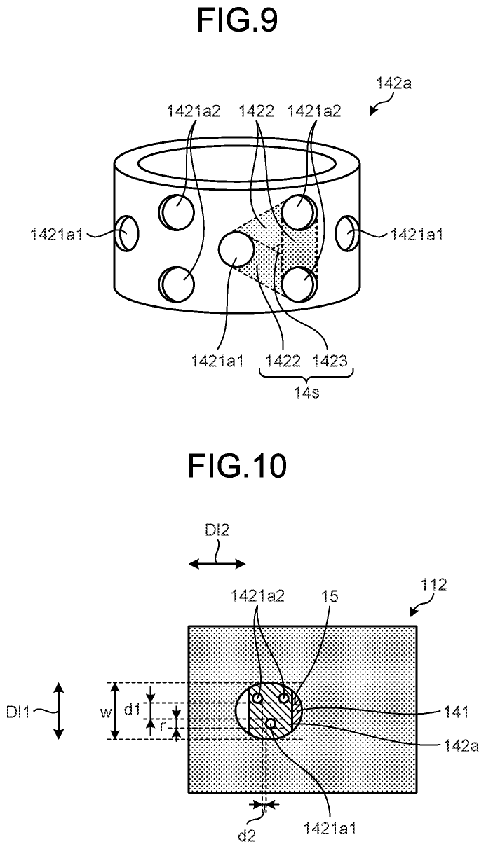

FIGS. 9 and 10 are diagrams for explaining a first modification of the embodiment. Specifically, FIG. 9 is a diagram corresponding to FIG. 4. FIG. 10 is a diagram corresponding to FIG. 5.

The first modification is different from the embodiment described above in the arrangement of the plurality of adjustment holes 1421 and has the same configuration as that of the embodiment described above except for the arrangement. In the first modification, the same components as those of the embodiment described above are denoted by the same reference numerals, and description thereof is omitted. Note that, in the following description, for convenience of description, the rigid portion 142 and the adjustment hole 1421 according to the first modification will be referred to as a rigid portion 142a and an adjustment hole 1421a (alternatively referred to first surface, second surface and/or recessed surface), respectively.

As illustrated in FIG. 9, the rigid portion 142a is provided with three rows of a plurality of adjustment holes 1421a arranged along the second direction DI2 while being spaced apart from each other by a predetermined distance along the first direction DI1. Among the three rows, the adjustment hole 1421a in the center row positioned at the center in the second direction DI2 is referred to as an adjustment hole 1421a1. Among the three rows, the adjustment holes 1421a in two both side rows positioned on both sides in the second direction DI2 are referred to as adjustment holes 1421a2. The adjustment hole 1421a1 in the center row is arranged at a position shifted in the first direction DI1 with respect to the adjustment holes 1421a2 in both side rows by half the predetermined distance described above. Note that, in FIG. 9, for convenience of description, only three boundary portions 1422 among the plurality of boundary portions 1422 are represented by dots.

The rigid portion 142a is provided at the distal end of the first elongated object 14. The rigid portion includes the outer peripheral surface 1422 or 1423, and the first surface (adjustment hole) 1421a1 or 1421a2 recessed from the outer peripheral surface 1422 or 1423 of the rigid portion 142a. Each of the outer peripheral surface 1422 or 1423 and the first surface 1421a1 or 1421a2 are exposed within the third opening. The rigid portion 142a comprises a second surface 1421a1 or 1421a2 recessed from the outer peripheral surface 1422 or 1423. The first surface 1421a or 1421a2 and the second surface 1421a or 1421a2 are separated from each other in the circumferential direction (DI1) of the rigid portion 142a.

Note that, even when the rigid portion 142a according to the first modification is employed, the light guide 14 can be positioned with respect to the second distal end component portion 112 by a method for producing similar to that of the embodiment described above.

As illustrated in FIG. 10, in the first modification, the machined hole 15 faces three adjustment holes 1421a (one adjustment hole 1421a1 and two adjustment holes 1421a2) and three boundary portions 1422 in a state after the light guide 14 is positioned with respect to the second distal end component portion 112 by the method for producing the endoscope 1. In other words, the rigid portion 142a is configured to have a relationship of W>d1+r, where W is the width dimension of the machined hole 15 in the first direction DI1 (the diameter dimension of the machined hole 15), r is the width dimension of the adjustment hole 1421a in the first direction DI1 (the diameter dimension of the adjustment hole 1421a), and d1 is the separation distance between the adjustment holes 1421a1 and 1421a2 adjacent to each other in the first direction DI1. The rigid portion 142a is also configured to have a relationship of W>d2+r, where W is the width dimension of the machined hole 15 in the second direction DI2 (the diameter dimension of the machined hole 15), r is the width dimension of the adjustment hole 1421a in the second direction DI2 (the diameter dimension of the adjustment hole 1421a), and d2 is the separation distance between the adjustment holes 1421a2 adjacent to each other in the second direction DI2.

According to the first modification described above, at least the following effects are obtained in addition to the same effects as those of the embodiment described above.

The rigid portion 142a according to the first modification is configured to have a relationship of W>d1+r, where W is the width dimension of the machined hole 15 in the first direction DI1 (the diameter dimension of the machined hole 15), r is the width dimension of the adjustment hole 1421a in the first direction DI1 (the diameter dimension of the adjustment hole 1421a), and d1 is the separation distance between the adjustment holes 1421a1 and 1421a2 adjacent to each other in the first direction DI1. The rigid portion 142a is also configured to have a relationship of W>d2+r, where W is the width dimension of the machined hole 15 in the second direction DI2 (the diameter dimension of the machined hole 15), r is the width dimension of the adjustment hole 1421a in the second direction DI2 (the diameter dimension of the adjustment hole 1421a), and d2 is the separation distance between the adjustment holes 1421a2 adjacent to each other in the second direction DI2. Therefore, when one adjustment hole 1421a of two adjustment holes 1421a1 and 1421a2 adjacent to each other is pushed in the first direction DI1 by the adjustment tool C to rotate the light guide 14 about the optical axis, the other adjustment hole 1421a is always exposed from the machined hole 15. In other words, the light guide 14 can be further rotated about the optical axis by using the other adjustment hole 1421a. When one adjustment hole 1421a of two adjustment holes 1421a1 and 1421a2 adjacent to each other is pushed in the second direction DI2 by the adjustment tool C to move the light guide 14 in the optical axis direction, the other adjustment hole 1421a is always exposed from the machined hole 15. In other words, the light guide 14 can be further moved in the optical axis direction by using the other adjustment hole 1421a. Therefore, the amounts of positioning about the optical axis and in the optical axis direction in the light guide 14 are increased, which further allows good positioning.

### Second Modification

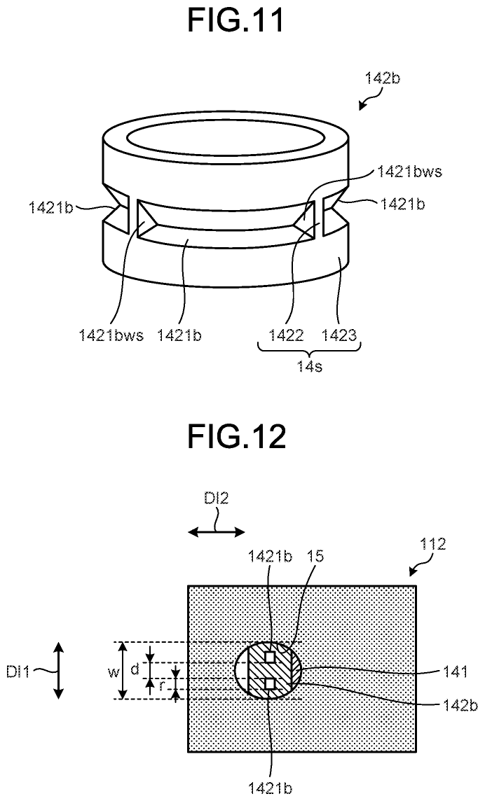

FIGS. 11 and 12 are diagrams for explaining a second modification of the embodiment. Specifically, FIG. 11 is a diagram corresponding to FIG. 4. FIG. 12 is a diagram corresponding to FIG. 5.

The second modification is different from the embodiment described above in the shape of the plurality of adjustment holes 1421 and has the same configuration as that of the embodiment described above except for the shape. In the second modification, the same components as those of the embodiment described above are denoted by the same reference numerals, and description thereof is omitted. Note that, in the following description, for convenience of description, the rigid portion 142 and the adjustment hole 1421 according to the second modification will be referred to as a rigid portion 142b and an adjustment hole 1421b (alternatively referred to first and/or second surfaces), respectively.

As illustrated in FIG. 11, the adjustment hole 1421b has a valley shape in which its opening has a rectangular shape, the area of the opening decreases toward the bottom, and the cross-section taken along a plane including the central axis of the rigid portion 142b has a V-shape. Note that the adjustment hole 1421b may have a valley shape in which the cross-section taken along a plane orthogonal to the central axis of the rigid portion 142b has a V-shape, or may have a shape having a bottom surface as in the embodiment described above without being limited to the valley shape. The opening of the adjustment hole 1421b may not be a rectangle but may be another polygon or an ellipse.

As in the adjustment hole 1421 described in the embodiment described above, the adjustment hole 1421b has a size into which the distal end CT of the adjustment tool C can be inserted. The distal end 18T of the screw 18 has a size incapable of being inserted into the adjustment hole 1421b.

The rigid portion 142b is provided at the distal end of the first elongated object 14. The rigid portion includes the outer peripheral surface 1422 or 1423, and the first surface (adjustment hole) 1421b or 1421bws recessed from the outer peripheral surface 1422 or 1423 of the rigid portion 142b. Each of the outer peripheral surface 1422 or 1423 and the first surface 1421b or 1421bws are exposed within the third opening. The rigid portion 142b comprises a second surface 1421b or 1421bws recessed from the outer peripheral surface 1422 or 1423. The first surface 1421b or 1421bws and the second surface 1421b or 1421bws are separated from each other in the circumferential direction (DI1) of the rigid portion 142b.

The first surface 1421 includes the side surfaces 1421b, the side surfaces form the angle θ of 90° to 135° relative to each other.

Note that, even when the rigid portion 142b according to the second modification is employed, the light guide 14 can be positioned with respect to the second distal end component portion 112 by a method for producing similar to that of the embodiment described above.

As illustrated in FIG. 12, in the first modification, the machined hole 15 faces the two adjustment holes 1421b and the boundary portion 1422 in a state after the light guide 14 is positioned with respect to the second distal end component portion 112 by the method for producing the endoscope 1. In other words, the rigid portion 142b is configured to have a relationship of W>d+r, where W is the width dimension of the machined hole 15 in the first direction DI1 (the diameter dimension of the machined hole 15), r is the width dimension of the adjustment hole 1421b in the first direction DI1, and d is the separation distance between the adjustment holes 1421b adjacent to each other in the first direction DI1.

According to the second modification described above, at least the following effects are obtained in addition to the same effects as those of the embodiment described above.

In the rigid portion 142b according to the second modification, the adjustment hole 1421b is formed in a valley shape. Therefore, even in a case where the machined hole 15 is small and the rigid portion 142b is hardly visible, when the adjustment tool C is inserted into the adjustment hole 1421b and moved, the adjustment tool C is guided to the side surface 1421bws along the valley. Therefore, even in the above case, the adjustment tool C can be easily brought into contact with the side surface 1421bws. In other words, positioning can be easily performed.

### Third Modification

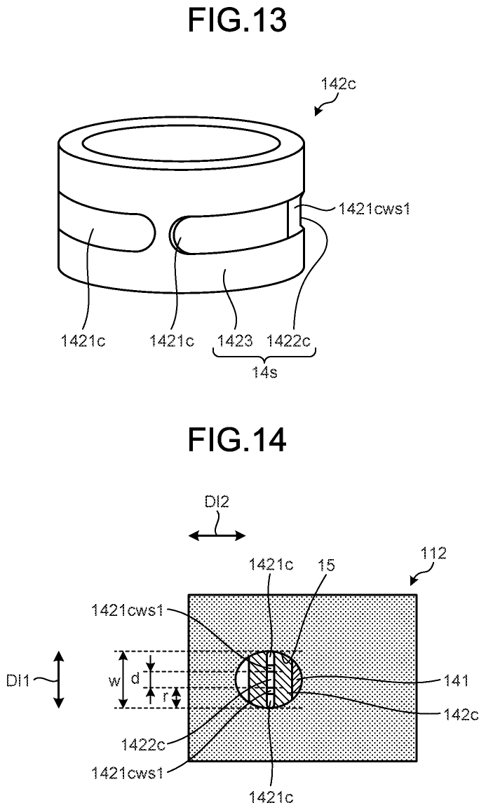

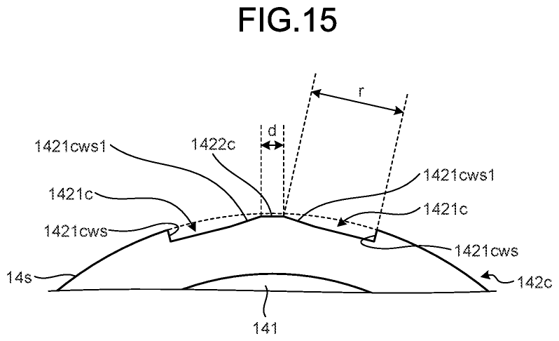

FIGS. 13 to 15 are diagrams for explaining a third modification of the embodiment. Specifically, FIG. 13 is a diagram corresponding to FIG. 4. FIG. 14 is a diagram corresponding to FIG. 5. FIG. 15 is a diagram of a rigid portion 142c viewed along the first direction DI1.

The third modification is different from the embodiment described above in the shape of the plurality of adjustment holes 1421 and the shape of the boundary portion 1422 and has the same configuration as that of the embodiment described above except for the shapes. In the third modification, the same components as those of the embodiment described above are denoted by the same reference numerals, and description thereof is omitted. Note that, in the following description, for convenience of description, the rigid portion 142, the adjustment hole 1421, and the boundary portion 1422 according to the third modification will be referred to as a rigid portion 142c, an adjustment hole 1421c (alternatively referred to first surface, second surface and/or recessed surface), and a boundary portion 1422c, respectively.

As illustrated in FIGS. 13 to 15, the adjustment hole 1421c has an opening having a substantially rectangular shape. In the rigid portion 142c, a plurality of sets of adjustment holes 1421c is arranged along the first direction DI1. The set of adjustment holes 1421c is two adjustment holes 1421c adjacent to each other. When attention is paid to the set of adjustment holes 1421c, a side surface 1421cws1 on the other adjustment hole 1421c side among side surfaces 1421cws constituting one adjustment hole 1421c is defined as an inclined surface 1421cws1. The inclined surface 1421cws1 is an inclined surface whose height from the central axis of the rigid portion 142c increases toward the other adjustment hole 1421c. In other words, as illustrated in FIG. 15, the inclined surface 1421cws1 is formed so as to intersect the first direction DI1 and the radial direction of the rigid portion 142c.

The boundary portion 1422c is formed such that the height in the radial direction of the rigid portion 142c is lower than the original position of the outer peripheral surface 14s (broken line in FIG. 15). In other words, the two adjustment holes 1421c and the boundary portion 1422c are formed by raising a part of the bottom of one large adjustment hole.

Similar to that shown in FIG. 7, the first surface 1421c includes the side surface 1421cws, 1421cws1 and the bottom surface, the side surface 1421cws, 1421cws1 and the bottom surface 1421c form the angle θ of 90° to 135° relative to each other.

As in the adjustment hole 1421 described in the embodiment described above, the adjustment hole 1421c has a size into which the distal end CT of the adjustment tool C can be inserted. The distal end 18T of the screw 18 has a size incapable of being inserted into the adjustment hole 1421c.

The rigid portion 142c is provided at the distal end of the first elongated object 14. The rigid portion includes the outer peripheral surface 1423, and the first surface (adjustment hole) 1421c recessed from the outer peripheral surface 1423 of the rigid portion 142c. Both of the outer peripheral surface 1422 or 1423 and the first surface 1421c are exposed within the third opening. The rigid portion 142c comprises a second surface 1421c recessed from the outer peripheral surface 1422 or 1423. The first surface 1421c and the second surface 1421c are separated from each other in the circumferential direction (DI1) of the rigid portion 142c. The rigid portion 142c includes a recessed surface 1421c, a raised surface 1422c adjacent to the recessed surface 1421c, a transition surface 1421cws1 separating from the recessed surface 1421c and the raised surface 1422c. The transition surface 1421cws1 forms a transition between the recessed surface 1421c and the raised surface 1422c. At least a portion of each of the recessed surface 1421c, the raised surface 1422c and the transition surface 1421cws1 are exposed within the third opening.

Note that, even when the rigid portion 142c according to the third modification is employed, the light guide 14 can be positioned with respect to the second distal end component portion 112 by a method for producing similar to that of the embodiment described above.

As illustrated in FIG. 14, in the third modification, the machined hole 15 faces the two adjustment holes 1421c and the boundary portion 1422c in a state after the light guide 14 is positioned with respect to the second distal end component portion 112 by the method for producing the endoscope 1. FIG. 14 illustrates a state in which the above-described set of adjustment holes 1421c faces the machined hole 15. In other words, the rigid portion 142c is configured to have a relationship of W>d+r, where W is the width dimension of the machined hole 15 in the first direction DI1 (the diameter dimension of the machined hole 15), r is the width dimension of the adjustment hole 1421c in the first direction DI1, and d is the separation distance between the adjustment holes 1421c adjacent to each other in the first direction DI1. In the state of FIG. 14, the separation distance d is the separation distance between the two inclined surfaces 1421cws1 in the first direction DI1, and is the width dimension of the boundary portion 1422c in the first direction DI1. The width dimension r is a length from a position where the height of the inclined surface 1421cws1 (height of the rigid portion 142c in the radial direction) is the highest in one adjustment hole 1421c of the above-described set of adjustment holes 1421c to a side surface 1421cws on a side spaced apart from the other adjustment hole 1421c.

Even when the rigid portion 142c according to the third modification described above is employed, the same effects as those of the embodiment described above are obtained.

### Other Embodiments

While the embodiments for carrying out the disclosure have been described above, the disclosure should not be limited only to the embodiments and the first to third modifications described above.

In the embodiment and the first to third modifications described above, the screw 18 has a size incapable of being inserted into the adjustment hole 1421 (1421a, 1421b, 1421c), but is not limited thereto. For example, the screw 18 may be formed to have a size capable of being inserted into the adjustment hole 1421 (1421a, 1421b, 1421c), and the screw 18 may be fixed in a state of being in contact with the inner surface of the adjustment hole 1421 (1421a, 1421b, 1421c).

For example, when the configuration described above is employed in the second modification described above, the distal end 18T of the screw 18 comes into contact with the valley portion of the adjustment hole 1421b, so that the light guide 14 can be stably fixed by the screw 18.

Further, for example, in a case where the configuration described above is employed in the third modification described above, the distal end 18T of the screw 18 may be brought into contact with the boundary portion 1422c.

In the embodiment and the first to third modifications described above, an elastic member may be provided at a portion of the distal end 18T that comes into contact with the rigid portion 142 (142a, 142b, 142c), and the elastic member may come into contact with and press both the outer peripheral surface 14s and the inner surface of the adjustment hole 1421 (1421a, 1421b, 1421c).

The fixing portion 18 inserted into the second channel 15, the fixing portion contacts the outer peripheral surface 1422 or 1423 so as to hold the rigid portion 142. The fixing portion 18 is configured to press the outer peripheral surface 1422 or 1423 of the rigid portion 142.

In the embodiment and the first to third modifications described above, the rigid portion 142 (142a, 142b, 142c) is provided in the first portion 1411 of the light guide 14, but may be provided over the entire length, including the bent portion 1410 and the second portion 1412.

In the embodiment and the first and second modifications described above, the plurality of adjustment holes 1421 (1421a, 1421b) is provided, but only one adjustment hole may be provided. In this case, the boundary portion 1422 corresponds to the entire outer peripheral surface of the rigid portion 142 (142a, 142b). Note that, similarly, in the third modification, only one set of adjustment holes 1421c may be provided.

Although the positioning of the light guide 14 has been described in the embodiment and the first to third modifications described above, other built-in objects such as an imaging unit and a treatment tool channel can be similarly positioned by employing a similar structure.

Although the positioning of the light guide 14 having the bent portion 1410 has been described in the embodiment and the first to third modifications described above, a light guide not having the bent portion 1410 can also be positioned by employing a similar structure.

According to the disclosure, it is possible to provide an endoscope capable of preventing a built-in object from being damaged when the built-in object is attached to the distal end portion of the insertion portion, and an endoscope producing method.
Example 1. An endoscope comprising:
   an insertion portion;
   a long first built-in object provided in the insertion portion;
   a rigid portion provided at a distal end of the first built-in object, the rigid portion including an outer peripheral surface about a longitudinal axis of the first built-in object, the outer peripheral surface including a plurality of hole portions; and
   a distal end main body provided at a distal end of the insertion portion, the distal end main body being configured to hold the first built-in object, wherein
   the distal end main body includes a first hole and a second hole communicating with the first hole,
   the second hole extends from a side surface of the distal end main body toward an inside of the distal end main body and faces the outer peripheral surface of the rigid portion, and
   the rigid portion is inserted into the first hole.
Example 2. The endoscope according to Example 1, wherein the plurality of hole portions is provided to be spaced apart from each other in a circumferential direction about the longitudinal axis.
Example 3. The endoscope according to Example 1, wherein the plurality of hole portions is provided to be spaced apart from each other in a direction of the longitudinal axis.
Example 4. The endoscope according to Example 2, wherein among side surfaces constituting the hole portions, a side surface serving as a boundary portion between the hole portions adjacent to each other is a surface formed so as to form an angle of 90° or more and 135° or less with respect to a bottom of each of the hole portions adjacent to each other.
Example 5. The endoscope according to Example 1, further comprising a fixing portion disposed in the second hole, the fixing portion being configured to press and fix at least one of a bottom of a hole portion and the outer peripheral surface of the rigid portion.
Example 6. The endoscope according to Example 5, wherein the fixing portion is configured to press a boundary portion between the hole portions adjacent to each other.
Example 7. The endoscope according to Example 6, wherein a distal end of the fixing portion has a size incapable of being inserted into each of the hole portions.
Example 8. The endoscope according to Example 5, wherein a distal end of the fixing portion has a size capable of being inserted into each of the hole portions.
Example 9. The endoscope according to Example 1, wherein
   the first built-in object includes a bent portion that is bent in a middle along the longitudinal axis, and
   the rigid portion is provided on a distal-end side of the bent portion.
Example 10. The endoscope according to Example 9, wherein
   the first built-in object is an illumination unit configured to emit light for illuminating a subject, and
   the longitudinal axis is an optical axis of the illumination unit.
Example 11. The endoscope according to Example 10, wherein the illumination unit is a light guide.
Example 12. The endoscope according to Example 1, wherein the second hole and a boundary portion between the hole portions adjacent to each other face each other.
Example 13. The endoscope according to Example 12, wherein the first built-in object is held by the distal end main body by being positioned such that the boundary portion faces the second hole.
Example 14. The endoscope according to Example 13, wherein each of the hole portions has a size into which a distal end of an adjustment tool used for positioning can be inserted.
Example 15. The endoscope according to Example 1, wherein the endoscope is configured to satisfy a relationship of W>d+r,
   where W is a width dimension of the second hole in one direction of a circumferential direction about the longitudinal axis and a direction of the longitudinal axis, r is a width dimension of each of the hole portions in the one direction, and d is a separation distance between the hole portions adjacent to each other in the one direction.
Example 16. The endoscope according to Example 1, wherein the second hole is formed so as not to interfere with a second built-in object, which is another built-in object other than the first built-in object.
Example 17. The endoscope according to Example 1, wherein the rigid portion is formed of a member that is more rigid than the built-in object.
Example 18. An endoscope producing method, comprising:
   inserting a distal end of a built-in object into a first hole of a distal end main body;
   inserting an adjustment tool into a second hole of the distal end main body; and
   performing positioning of the built-in object with respect to the distal end main body by applying a load to a rigid portion of the built-in object with the adjustment tool.
Example 19. The endoscope producing method according to claim 18, wherein the positioning is performed such that a boundary portion between hole portions adjacent to each other in the rigid portion is arranged at a position visible from the second hole.
Example 20. An endoscope comprising:
   an insertion portion;
   a long built-in object provided in the insertion portion;
   a rigid portion provided at a distal end of the built-in object, the rigid portion including an outer peripheral surface about a longitudinal axis of the built-in object, the outer peripheral surface including a plurality of hole portions; and
   a distal end main body provided at a distal end of the insertion portion, the distal end main body being configured to hold the built-in object, wherein
   the distal end main body includes a first hole and a second hole communicating with the first hole,
   the second hole extends from a side surface of the distal end main body toward an inside of the distal end main body,
   the rigid portion is inserted into the first hole, and
   the endoscope is configured to satisfy a relationship of W>d+r,
   where W is a width dimension of the second hole in one direction of a circumferential direction about the longitudinal axis and a direction of the longitudinal axis, r is a width dimension of each of the hole portions in the one direction, and d is a separation distance between the hole portions adjacent to each other in the one direction.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An insertion portion of an endoscope comprising:
a distal end portion comprising:
an exterior surface,
a first channel including a first opening on the exterior surface, and
a second channel including a second opening on the exterior surface and a third opening on the first channel,
an elongated object inserted into the first channel;
a rigid portion provided at a distal end of the first elongated object, the rigid portion including:
an outer peripheral surface, and
a first surface recessed from the outer peripheral surface of the rigid portion,
wherein both of the outer peripheral surface and the first surface are exposed within the third opening.

2. The insertion portion according to claim 1, the rigid portion comprising a second surface recessed from the outer peripheral surface.

3. The insertion portion according to claim 2, wherein the first surface and the second surface are separated from each other in a circumferential direction of the rigid portion.

4. The insertion portion according to claim 3, wherein
the rigid portion is configured to satisfy a relationship of W>d+r,
where W is a width dimension of the second opening, r is a width dimension of each of the first surface and the second surface, and d is a distance between the first surface and the second surface.

5. The insertion portion according to claim 1, wherein the first surface includes a side surface and a bottom surface, the side surface and the bottom surface form an angle of 90° to 135° relative to each other.

6. The insertion portion according to claim 1, further comprising a fixing portion inserted into the second channel, the fixing portion contacting the outer peripheral surface so as to hold the rigid portion.

7. The insertion portion according to claim 6, wherein the fixing portion is configured to press the outer peripheral surface of the rigid portion.

8. The insertion portion according to claim 7, wherein the fixing portion includes a distal end having a size greater than a size of an opening of the recessed surface on the outer peripheral surface.

9. The insertion portion according to claim 1, wherein
the first elongated object includes a distal portion at a distal-end side that is bent relative to a proximal portion of the first elongated object, and
the rigid portion is provided on the distal portion.

10. The insertion portion according to claim 9, wherein
the first elongated object is an illumination unit configured to emit light for illuminating a subject.

11. The insertion portion according to claim 10, wherein the illumination unit is a light guide.

12. The insertion portion according to claim 1,
wherein the exterior surface includes an outer surface, a distal end surface and an inclined surface between the outer surface and the distal end surface,
wherein the first opening is provided on the inclined surface, and the second opening is provided on the outer surface.

13. The insertion portion according to claim 1, wherein a first longitudinal axis of the first channel intersects with a second longitudinal axis of the second channel.

14. The insertion portion according to claim 1, wherein the second channel extends linearly.

15. The insertion portion according to claim 1, wherein the rigid portion is formed of a first material having a rigidity greater than a rigidity of a second material forming the elongated object.

16. The insertion portion according to claim 1, wherein the rigid portion includes:
a raised surface adjacent to one or more of the first surface and the second surface, and
a transition surface separate from the first surface and the raised surface, the transition surface forming a transition between the raised surface and one or more of the first surface and the second surface,
wherein at least a portion of each of the recessed surface, the raised surface and the transition surface are exposed within the third opening.

17. An endoscope comprising:
the insertion portion according to claim 1.

18. A method of assembling an insertion portion of an endoscope, the method comprising:
inserting a rigid portion onto a distal end of an elongated object;
inserting the elongated object into a first channel of a distal end portion;
positioning the rigid portion to a first opening of the first channel, the first opening communicating the first channel with a second channel of the distal end portion;
positioning one of a first surface recessed from an outer peripheral surface of the rigid portion at the first opening;
inserting an adjustment tool into the second channel; and
positioning of the elongated object with respect to the distal end portion by applying a load to the first surface through the second channel with the adjustment tool.

19. The method according to claim 16, further comprising,
positioning an outer peripheral surface of the rigid portion at the first opening.

20. An insertion portion of an endoscope comprising:
a distal end portion comprising:
an exterior surface,
a first channel including a first opening on the exterior surface, and
a second channel including a second opening on the exterior surface and a third opening on the first channel,
an elongated object inserted into the first channel;
a rigid portion provided at a distal end of the first elongated object, the rigid portion including:
a recessed surface,
a raised surface adjacent to the recessed surface; and
a transition surface separate from the recessed surface and the raised surface, the transition surface forming a transition between the recessed surface and the raised surface;
wherein at least a portion of each of the recessed surface, the raised surface and the transition surface are exposed within the third opening.
